(19) United States
(12) Patent Application Publication
(10) Pub. No.: US 2021/0298596 A1
(43) Pub. Date: Sep. 30, 2021

(54) CORNEA PROJECTION DEVICE FOR OPHTHALMIC SLIT LAMP MICROSCOPE

(71) Applicant: Yi Wang, Chong Qing (CN)

(72) Inventor: Yi Wang, Chong Qing (CN)

(21) Appl. No.: 16/827,761

(22) Filed: Mar. 24, 2020

(51) Int. Cl. A61B 3/135

(52) U.S. Cl. CPC A61B 3/135

(57) ABSTRACT

An additional cornea projection device for ophthalmic slit lamp microscope, comprising: a cornea projection device, a slit lamp microscope optical body, a curved arm and the connecting part. The cornea projection device is connected to the upper or lower part of the optical body of the slit lamp microscope, or to the left or right side of the curved arm. When the cornea projection device is not in use, it can be rotated to a non-working position while being kept connected, and there is no obstacle to the normal use of the slit light source projection device of the ophthalmic slit lamp microscope. An ophthalmic slit lamp microscope with a cornea projection device has a simple overall structure, reliable performance, low cost, obvious advantages, convenient for production, debugging and maintenance.

### TECHNICAL FIELD

The present invention relates to the field of medical equipment, and in particular, to cornea projection device for the ophthalmic slit lamp microscope.

### BACKGROUND TECHNOLOGY

Ophthalmic slit lamp microscope is an indispensable instrument for ophthalmic medical. The cornea projection device is connected to the slit lamp microscope, which can be connected to the hole in the rotating shaft(the hole for focusing rod), or to the upper part of the optical body of the lamp microscope, or to the lower part of the optical body of the lamp microscope, or to the left and right sides of the curved arm of the slit lamp microscope.

The cornea projection device is installed in the hole of the rotating axis of the slit lamp microscope, the advantage is that the interface is readily available, and there is no need to modify the slit lamp microscope. The disadvantages are:

a. When observe the ocular surface with slit lamp microscope through the hole in the center of the cornea projection device, the cornea projection device also needs to be manually rotated to the corresponding angle simultaneously when rotating the optical body of the slit lamp microscope, since the device is not connected to the optical body. It is inconvenient.

b. The hole in the rotating axis of the slit lamp optical body is generally a focusing rod interface, and the interface is occupied by the cornea projection device.

c. Due to the structure of the cornea projection device, it will interfere with the motion of the slit lamp, the original function of the slit lamp microscope cannot be used at the same time, so the cornea projection device must be removed when using the slit lamp function.

d. The control wiring of the cornea projection device is exposed, and it must be installed together with the cornea projection device when in use. And the cornea projection device is stored in the storage box together with the wiring when not in use. Frequent installation and removal will easily cause the connection to be unreliable. At the same time, the exposed wiring is also easily broken, and the appearance is not neat.

The cornea projection device is connected to the upper part of the slit lamp microscope optical body. The advantages are:

a. The cornea projection device rotates synchronously with the slit lamp microscope optical body, which can be conveniently carried out multi-angle observation of the cornea.

b. When not in use, turn out of the working position without affecting the other functions of the slit lamp microscope, and you can rotate the device left or right, which will not affect the operation and view of the observer.

c. The control wiring is hidden inside the optical body. And the whole machine use hidden wiring design, the appearance is neat and aesthetic.

The disadvantage is that the hidden wiring passes through the inside of the optical body, and some parts need to be modified.

The cornea projection device is installed at the lower part of the optical body of the slit lamp microscope. The advantage are almost the same as when the cornea projection device is connected to the upper part of the optical body, but does not need to modify the optical parts because the wiring easily along the curved arm. There are almost no disadvantages.

The cornea projection device is installed on the left (or right) side of the curved arm of the slit lamp microscope, and has the same advantages as the cornea projection device is connected to the lower part of the optical body. The disadvantage is that it can only be rotated out of the working position on one side. For example, it cannot be rotated to the right when the device is connected to the left side of the curved arm. Because one side of the device is blocked by the curved arm, it can only be rotated with the left hand of the operator, which is inconvenient for the people who usually use the right hand. At the same time, the rightward rotation cannot be performed. The device can only be rotated to the left of the curved arm. It will impact the operation and view of the left side of slit lamp microscope. It has the similar situation if the device is connected on the right side of the curved arm.

To sum up, it is not advisable to connect the cornea projection device to the hole of the rotating axis of the slit lamp microscope. It is advisable that the cornea projection device is connected to the lower part of the optical body of the slit lamp microscope, or to the upper part of the optical body of the slit lamp microscope, or to the left (or right) side of the curved arm of the slit lamp microscope. Therefore, an additional cornea projection device for the ophthalmic slit lamp microscope is provided, which is connected to the upper or lower part of the optical body of the slit lamp microscope, or left (or right) side of the curved arm.

### CONTENTS OF THE INVENTION

In view of solving the shortcomings of the existing technology, the invention proposes an additional cornea projection device for the ophthalmic slit lamp. The specific technical solution is as follows:

An additional cornea projection device of ophthalmic slit lamp, comprising: a cornea projection device (1), a slit lamp microscope optical body (2), and a slit lamp curved arm (4).

The cornea projection device (1) can be connected to the lower part of the optical body (2) of the slit lamp microscope, or to the upper part of the optical body (2) of the slit lamp microscope, or to both sides of the curved arm (4) of the slit lamp microscope, moving with the optical body of the slit lamp microscope (2).

In order to better implement the invention, it may further be:

When the cornea projection device is not in use, it can be rotated to a non-working position while being kept connected, and there is no obstacle to the normal use of the slit light source projection device (3) of the ophthalmic slit lamp microscope.

The beneficial effects of the invention are: the overall structure is simple, convenient for large-scale production, reliable performance, low cost, and obvious advantages. The cornea projection device can synchronously rotate with the slit lamp optical body, which can perform multi-angle observation of the cornea. the cornea projection device is put into the working position when in use, and turned out of the working position when not in use, without affecting other functions of the slit lamp. The wiring of the cornea projection device is hidden and has a clean and aesthetic appearance. The production, debugging and maintenance of the cornea projection device are very convenient.

### DESCRIPTION OF THE FIGURES

FIG. 1 is an overall structural diagram of the present invention.

FIG. 2 is a sectional view of the present invention.

FIG. 3 is a schematic diagram of use.

The reference numerals are:

1-cornea projection device.

2-slit lamp microscope optical body.

3-slit light source projection device.

4-curved arm.

Concrete Implementing Scheme

The following describes the better implementation of the present invention in detail with reference to the figures, so that the advantages and features of the present invention can be more easily understood by those skilled in the art, and the protection scope of the present invention is more clearly defined.

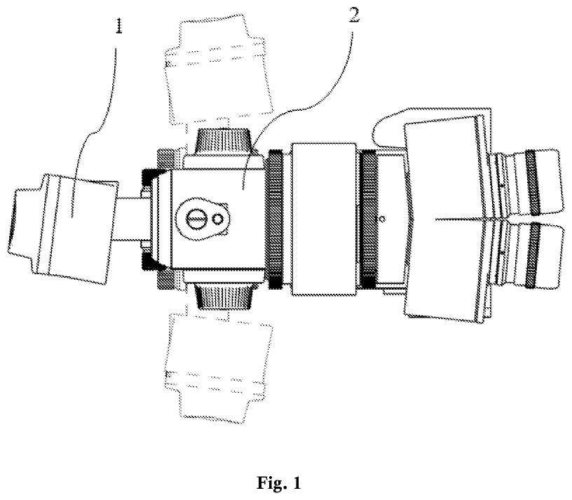

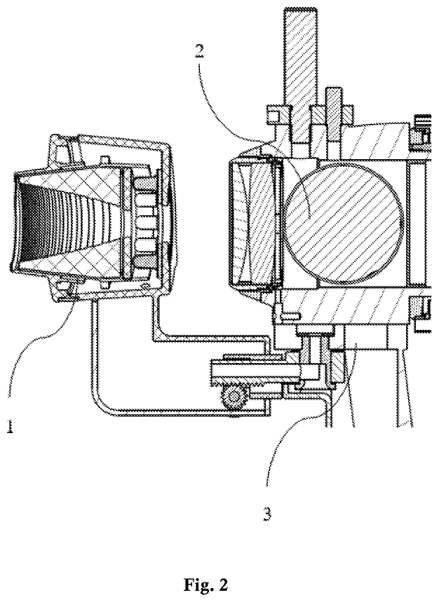

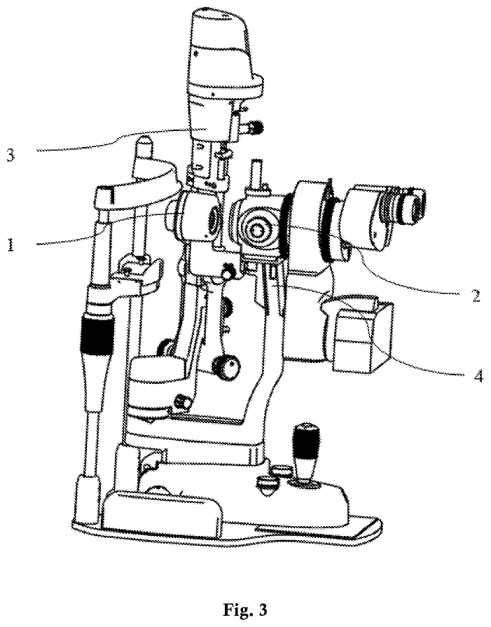

As shown in FIG. 1 to FIG. 3, an additional cornea projection device for the ophthalmic slit lamp microscope, comprising: a cornea projection device (1), a slit lamp microscope optical body (2), a slit light source projection device (3), and a curved arm (4). The cornea projection device (1) is connected to the lower part of the optical body (2) of the slit lamp microscope, the upper part of the optical body (2) of the slit lamp microscope, and the left or right side of the curved arm (4) of the slit lamp microscope, and moves together with the optical body (2) of the slit lamp microscope.

The cornea projection device (1) can be rotated to a non-working position while being kept connected when not in use, and there is no obstacle to the normal use of the slit light source projection device (3) of the ophthalmic slit lamp microscope.

We claim:

1. An additional cornea projection device for the ophthalmic slit lamp microscope, is characterized in that it comprise a cornea projection device, an optical body of the slit lamp microscope, and a curved arm of the slit lamp microscope. The cornea projection device can be connected to the lower part of the optical body part of the slit lamp microscope, or the upper part of the optical body part of the slit lamp microscope, or both sides of the curved arm of the slit lamp microscope, and can be moved with the slit lamp microscope optical body.

2. The cornea projection device, according to claim 1, is characterized in that the cornea projection device can be rotated to a non-operating position while it is not in use, and all functions of the slit lamp microscope are not affected in such state.
